(19) United States
(12) Patent Application Publication
(10) Pub. No.: US 2026/0117286 A1
(43) Pub. Date: Apr. 30, 2026

(54) SPATIAL ANALYSIS OF RNA METHYLATION

(71) Applicant: 10x Genomics, Inc., Pleasanton, CA (US)

(72) Inventors: Layla Katiraee, Castro Valley, CA (US); Zachary Bent, Pleasanton, CA (US)

(21) Appl. No.: 18/946,306

(22) Filed: Nov. 13, 2024

Related U.S. Application Data
(60) Provisional application No. 63/598,340, filed on Nov. 13, 2023.

(51) Int. Cl. C12Q 1/6841

(52) U.S. Cl. CPC C12Q 1/6841

(57) ABSTRACT

Provided herein are methods of identifying methylation status of a nucleic acid, e.g., RNA in a biological sample. Also provided herein are methods for identifying the methylation status of RNA in a biological sample with spatial technology to identify the location of a methylated RNA in the biological sample.

### CROSS-REFERENCE TO RELATED APPLICATIONS

This application claims priority to U.S. Provisional Patent Application No. 63/598,340, filed Nov. 13, 2023, the contents of which are incorporated herein by reference in their entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provide substantial analyte data for dissociated tissue (i.e., single cells), but fail to provide information regarding the position of the single cell in a parent biological sample (e.g., tissue sample).

The presence of N⁶-methyladenosine (m⁶A) in mRNAs is a regulated modification that is required for specific developmental processes and m⁶A modifications influence cell differentiation and other essential processes. Comprehensive mapping of m⁶A, aimed at elucidating the multitude of roles served by the modification, remains challenging. Therefore, there exists a need for methods of spatial analysis of RNA methylation status, e.g., m⁶A modifications of mRNAs in biological samples.

### SUMMARY

This disclosure provides methods of analysis that allow for identification of RNA methylation status, e.g., spatial analysis methods. The methods provided herein are applicable to normal physiological conditions, development and stem-cell studies, and in pathophysiological settings such as cancer and autoimmune diseases including, e.g., rheumatoid arthritis, lupus, multiple sclerosis (MS), and psoriasis. The role of m⁶A modification in autoimmune diseases is reviewed in, e.g., Wang Y, et al. The Emerging Role of m6A Modification in Regulating the Immune System and Autoimmune Diseases. Front Cell Dev Biol. 2021 Nov. 16; 9:755691, which is incorporated by reference in its entirety. For example, in one embodiment, the RNA methylation status (and changes of the same) of a tissue sample can be examined. And, because the location of the methylated RNA (or complement thereof) can be identified using spatial analysis methods (e.g., RNA-templated ligation) as disclosed herein, changes to the methylation status can be identified. In some instances, the methods disclosed herein can also be combined with imaging techniques that provide a correlation between a particular location of an image (e.g., location of a tumor in a biological sample) and both gene expression and RNA methylation status at that location. Thus, the disclosure provides methods of identifying the location of methylated RNA, e.g., N⁶-methyladenosine modifications of mRNA, in a biological sample.

In a first aspect, the disclosure provides methods for identifying methylation status of an RNA molecule in a biological sample, the methods including: (a) deaminating the RNA molecule in the biological sample by converting one or more non-methylated adenosines in the RNA molecule to inosines, thereby generating deaminated RNA; (b) hybridizing the deaminated RNA to a capture domain of a capture probe in a plurality of capture probes comprised in an array, wherein the capture probe further includes a spatial barcode; and (c) determining sequences of (i) the spatial barcode, or a complement thereof, and (ii) all or part of the deaminated RNA, or a complement thereof, and using the determined sequences of (i) and (ii) to identify the methylation status of the RNA molecule in the biological sample.

In another aspect, the disclosure provides methods for identifying methylation status of RNA in a biological sample, the methods including: (a) contacting the biological sample with a first substrate; (b) permeabilizing the biological sample; (c) deaminating the RNA in the biological sample by converting one or more non-methylated adenosines in the RNA to inosines, thereby generating deaminated RNA; (d) contacting the biological sample with a plurality of probes including a first probe and a second probe, wherein the first probe or the second probe hybridize to the deaminated RNA and wherein: the first probe and the second probe include a sequence complementary to a first sequence of the deaminated RNA; and the second probe includes a sequence complementary to a capture domain of a capture probe affixed to an array, wherein the capture probe further includes a spatial barcode; (e) ligating the first probe and the second probe, thereby generating a ligation product; (f) hybridizing the ligation product to the capture domain of the capture probe; and (g) determining sequences of (i) the spatial barcode, or a complement thereof, and (ii) all or part of the ligation product, or a complement thereof, and using the determined sequences of (i) and (ii) to identify the methylation status of the RNA in the biological sample.

In some embodiments, the array is on the first substrate. In some embodiments, the array is on a second substrate.

In some embodiments, the methods further include: aligning the first substrate with the second substrate, wherein a portion of the biological sample is aligned with at least a portion of the array; and releasing the ligation product from the deaminated RNA. In some embodiments, the methods further include migrating the ligation product from the biological sample to the array. In some embodiments, the first sequence of the deaminated RNA includes inosine and/or adenosine, preferably wherein the adenosine is N⁶-methyladenosine.

In another aspect, the disclosure provides methods for determining the location of a methylated RNA molecule in a biological sample, the method including: (a) contacting the biological sample with an adenosine deaminase such that one or more non-methylated adenosines in the RNA molecule is converted to inosine, thereby generating a deaminated RNA molecule; (b) hybridizing the deaminated RNA molecule to a capture domain of a capture probe of an array including a plurality of capture probes, wherein the capture probe further includes a spatial barcode; (c) extending the capture probe using the deaminated RNA molecule as a template, thereby generating an extended capture probe; and (d) determining sequences of (i) the spatial barcode, or a complement thereof, and (ii) all or part of the extended capture probe, or a complement thereof, and using the determined sequences of (i) and (ii) to determine the location of the methylated RNA molecule in the biological sample.

In another aspect, the disclosure provide methods for determining the location of a methylated RNA molecule in a biological sample, the methods including: (a) providing the biological sample mounted on a first substrate; (b) contacting the biological sample with an adenosine deaminase such that one or more non-methylated adenosines in the RNA molecule is converted to inosine, thereby generating a deaminated RNA molecule; (c) contacting the biological sample with a plurality of probes including a first probe and a second probe, wherein the first probe or the second probe hybridize to the deaminated RNA molecule and wherein: the first probe and the second probe include a sequence complementary to a first sequence of the deaminated RNA molecule, wherein the first sequence of the deaminated RNA molecule includes inosine and/or adenosine; and the second probe includes a sequence complementary to a capture domain of a capture probe of an array including a plurality of capture probes, wherein the capture probe further includes a spatial barcode; (d) ligating the first probe and the second probe, thereby generating a ligation product; (e) hybridizing the ligation product to the capture domain of the capture probe; and (f) determining sequences of (i) the spatial barcode, or a complement thereof, and (ii) all or part of the ligation product, or a complement thereof, and using the determined sequences of (i) and (ii) to determine the location of the methylated RNA molecule in the biological sample.

In some embodiments, the array including a plurality of capture probes is on a second substrate, with the methods further including: aligning the first substrate with the second substrate, wherein a portion of the biological sample is aligned with at least a portion of the array; and releasing the ligation product from the deaminated RNA molecule.

In some embodiments, the methods further include migrating the ligation product from the biological sample to the array. In some embodiments, the methylated RNA molecule includes one or more methylated adenosines, optionally wherein the one or more methylated adenosines is N⁶-methyladenosine. In some embodiments, the one or more methylated adenosines remains methylated upon contacting the biological sample with the adenosine deaminase.

In some embodiments, the methods further include permeabilizing the biological sample. In some embodiments, the biological sample is disposed on the array. In some embodiments, the deaminating is performed with the biological sample disposed on the array. In some embodiments, the biological sample is optionally treated with a DNase. In some embodiments, the RNA molecule or methylated RNA molecule is mRNA. In some embodiments, the deaminating includes treating the biological sample with an adenosine deaminase. In some embodiments, the adenosine deaminase is tRNA-specific adenosine deaminase (TadA) or variant or equivalent thereof. In some embodiments, the adenosine deaminase is TadA8.20.

In some embodiments, the capture probes include a poly(T) sequence. In some embodiments, capture probes further include one or more functional domains, a unique molecular identifier, a cleavage domain, or combinations thereof.

In some embodiments, the biological sample is a tissue sample. In some embodiments, the tissue sample is a tissue section. In some embodiments, the tissue sample is a fixed tissue sample. In some embodiments, the fixed tissue sample is a formalin fixed paraffin embedded (FFPE) tissue sample. In some embodiments, the FFPE tissue is deparaffinized and decrosslinked, optionally prior to step (a). In some embodiments, the fixed tissue sample is a formalin fixed paraffin embedded cell pellet. In some embodiments, the tissue sample is a fresh frozen tissue sample. In some embodiments, the tissue sample is fixed and stained, optionally prior to step (a). In some embodiments, the tissue sample is stained using immunofluorescence, immunohistochemistry, hematoxylin or eosin (H&E).

In some embodiments, the determining includes sequencing. In some embodiments, the sequencing includes high-throughput sequencing.

In some embodiments, the permeabilizing includes use of a permeabilization reagent including a protease. In some embodiments, the protease includes pepsin. In some embodiments, the protease includes proteinase K. In some embodiments, the permeabilization reagent further includes one or more of a lipase, a DNase, an RNase, a detergent, or combinations thereof, preferably wherein the permeabilization reagent further includes DNase.

In some embodiments, the methods further include extending the capture probe using the ligation product as a template, or extending the ligation product using the capture probe as a template, optionally wherein the extending includes use of a polymerase.

In some embodiments, the methods further include extending the capture probe using the deaminated RNA as a template, thereby generating an extended capture probe. In some embodiments, the determining includes determining a sequence of the extended capture probe, or an amplicon or a complement thereof. In some embodiments, the RNA molecule includes one or more methylated adenosines, wherein the one or more methylated adenosines is N⁶-methyladenosine, and optionally wherein the one or more methylated adenosines remains methylated upon contacting the biological sample with an adenosine deaminase. In some embodiments, the biological sample is a formalin fixed paraffin embedded (FFPE) tissue section, and the method further includes decrosslinking the tissue section, incorporating a poly(A) sequence at a 3′end of the deaminated RNA, or a combination thereof.

In another aspect, the disclosure provide kits including: (a) an array including a plurality of capture probes, wherein each of the capture probes includes a (i) a spatial barcode, (ii) a capture domain, and (iii) one or more functional domains; and (b) an adenosine deaminase.

In some embodiments, the kits further include a polymerase. In some embodiments, the polymerase includes one of a T7 RNA polymerase, a T3 RNA polymerase, or an SP6 RNA polymerase. In some embodiments, the kits further include one or more permeabilization reagents. In some embodiments, the one or more permeabilization reagents include one or more of a protease, a lipase, a DNase, an RNase, a detergent, or combinations thereof. In some embodiments, the protease includes pepsin or proteinase K.

In another aspect, the disclosure provides methods for identifying methylation status of RNA in a biological sample, the methods including: (a) providing the biological sample mounted on a first substrate; (b) contacting the biological sample with an adenosine deaminase such that one or more non-methylated adenosines in the RNA is converted to inosine, thereby generating a deaminated RNA; (c) contacting the biological sample with a plurality of probes including a first probe and a second probe, wherein the first probe or the second probe hybridize to the deaminated RNA, and wherein the first probe and the second probe include a sequence complementary to a first sequence of the deaminated RNA molecule, wherein the first sequence of the deaminated RNA molecule includes inosine and/or adenosine; (d) ligating the first probe and the second probe, thereby generating a ligation product; (e) hybridizing a padlock probe to the ligation product; (f) circularizing the padlock probe using e.g., a ligase, thereby generating circularized padlock probe; (g) optionally amplifying the circularized padlock probe, thereby generating an amplified padlock probe product; (h) detecting the amplified padlock probe product using one or more detection probes, optionally wherein the one or more detection probes collectively include one or more fluorophores.

In another aspect, the disclosure provides methods for identifying methylation status of RNA in a biological sample, the method including: (a) providing the biological sample mounted on a first substrate; (b) contacting the biological sample with an adenosine deaminase such that one or more non-methylated adenosines in the RNA is converted to inosine, thereby generating a deaminated RNA; (c) contacting the biological sample with a padlock probe, wherein the padlock probe hybridizes to the deaminated RNA; (d) circularizing the padlock probe using, e.g., a ligase, thereby generating circularized padlock probe; (e) optionally amplifying the circularized padlock probe, thereby generating an amplified padlock probe product; and (f) detecting the amplified padlock probe product using one or more detection probes, optionally wherein the one or more detection probes collectively include one or more fluorophores.

In some embodiments, the padlock probe includes a first sequence at its 5′ end and a second sequence at its 3′ end, wherein the first sequence is substantially complementary to a first portion of the deaminated RNA, and the second sequence is substantially complementary to a second portion of the deaminated RNA. In some embodiments, the padlock probe further includes a backbone sequence, optionally wherein the backbone sequence is disposed between the first sequence at its 5′ end and the second sequence at its 3′ end; optionally wherein a portion of the backbone sequence is substantially complementary to an amplification primer. In some embodiments, the backbone sequence includes a backbone barcode sequence, optionally wherein the backbone barcode sequence is unique to and/or is used to identify the RNA. In some embodiments, circularizing the padlock probe includes ligating the first sequence and the second sequence. In some embodiments, amplifying the circularized padlock probe includes rolling circle amplification (RCA), optionally using a primer that is substantially complementary to a portion of the backbone sequence of the padlock probe.

In some embodiments, the RNA includes one or more methylated adenosines, optionally wherein the one or more methylated adenosines is N⁶-methyladenosine. In some embodiments, the one or more methylated adenosines remains methylated upon contacting the biological sample with the adenosine deaminase. In some embodiments, the RNA is mRNA. In some embodiments, the adenosine deaminase is TadA or variant or equivalent thereof, optionally wherein the adenosine deaminase is TadA8.20. In some embodiments, the biological sample is a tissue sample or tissue section, optionally a fixed tissue section or fresh frozen tissue section.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

FIG. 2A shows a perspective view of an exemplary sample handling apparatus in a closed position.

FIG. 2B shows a perspective view of an exemplary sample handling apparatus in an open position.

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

FIG. 4A shows a side view of the angled closure workflow.

FIG. 4B shows a top view of the angled closure workflow.

FIG. 5 is a schematic diagram showing an example of a barcoded capture probe, as described herein.

FIG. 6 shows a schematic illustrating a cleavable capture probe.

FIG. 7 shows exemplary capture domains on capture probes.

FIG. 8 shows an exemplary arrangement of barcoded features within an array.

FIG. 9A shows an exemplary workflow for performing templated capture and producing a ligation product, and FIG. 9B shows an exemplary workflow for capturing a ligation product from FIG. 9A on a substrate.

FIG. 10 is a schematic diagram of an exemplary analyte capture agent.

FIG. 11 is a schematic diagram depicting an exemplary interaction between a feature-immobilized capture probe 1124 and an analyte capture agent 1126.

FIG. 12A is an exemplary schematic of a tissue section placed on a slide, stained and imaged prior to detection of methylation status of RNA within the tissue section.

FIG. 12B is a schematic of deamination of RNA within the tissue section by a deaminating agent, wherein non-methylated adenosines in the RNA in the tissue section are globally deaminated and converted to inosine, while N⁶-methyladenosines are not deaminated.

FIG. 12C is a schematic showing how an array of capture probes is used to capture (i) the RNA analytes in the tissue section or (ii) ligation products of a first and second probe hybridized to RNA analytes in the tissue section to facilitate capture of RNA or a proxy thereof.

FIG. 12D is a heatmap representing the percentage or abundance of methylated adenosines in the RNA within the tissue section as detected by methods disclosed herein.

### DETAILED DESCRIPTION

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788, WO2017/144338, and WO2016/057552; Non-patent literature references Rodriques et al., Science 363(6434): 1463-1467, 2019; Lee et al., Nat. Protoc. 10(3):442-458, 2015; Trejo et al., PLoS ONE 14(2): e0212031, 2019; Chen et al., Science 348(6233):aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I) (b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archaeon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via eosin and/or hematoxylin), imaged, destained (e.g., via HCl), or a combination thereof. In some embodiments, when a fresh frozen tissue sample is fixed in methanol, the sample is treated with isopropanol prior to being stained (e.g., via eosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via eosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology and biomolecules. PAXgene provides a two-reagent fixative system in which tissue is firstly fixed in a solution containing methanol and acetic acid, then stabilized in a solution containing ethanol. See, Ergin B. et al., J Proteome Res. 2010 Oct. 1; 9(10):5188-96; Kap M. et al., PLoS One.; 6(11):e27704 (2011); and Mathieson W. et al., Am J Clin Pathol.; 146(1):25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene. In some embodiments, the biological sample, e.g., the tissue sample, is fixed, for example in methanol, acetone, acetone-methanol, PFA, PAXgene, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or eosin. Non-limiting examples of stains include histological stains (e.g., hematoxylin and/or eosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I) (d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Publication No. WO2020/176788 and/or U.S. Patent Application Publication No. 2020/0277663, which is herein incorporated by reference.

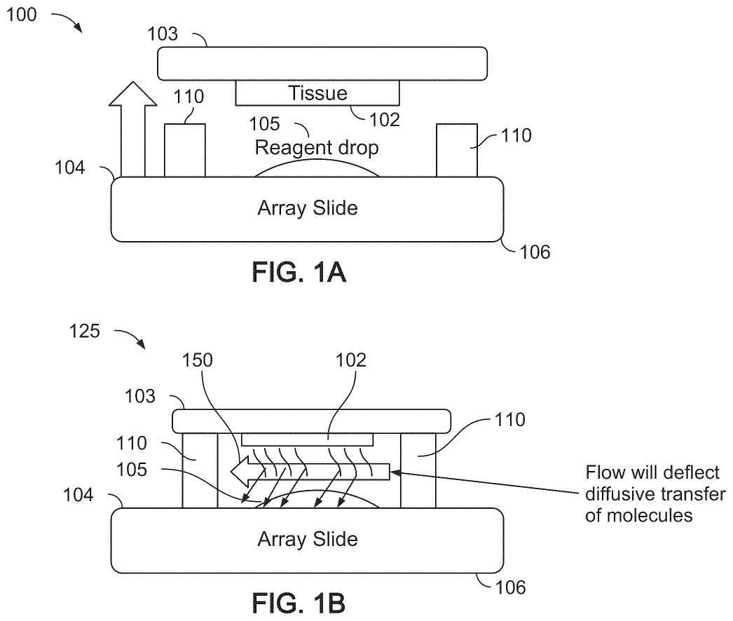

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the array of capture probes is a second substrate. In this configuration, one or more analytes or analyte derivatives (e.g., intermediate agents; e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference. FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

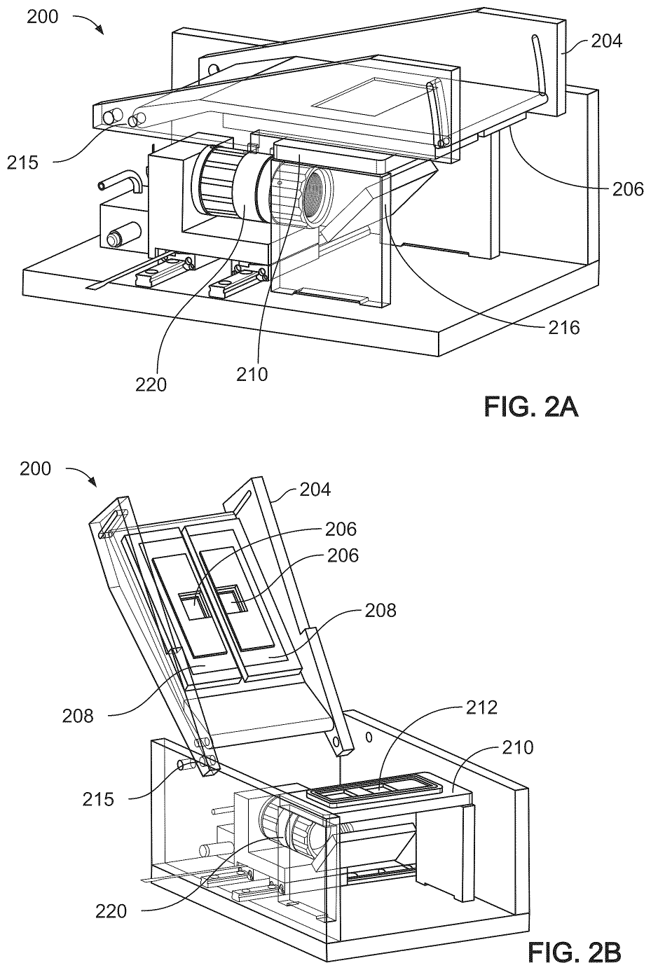

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

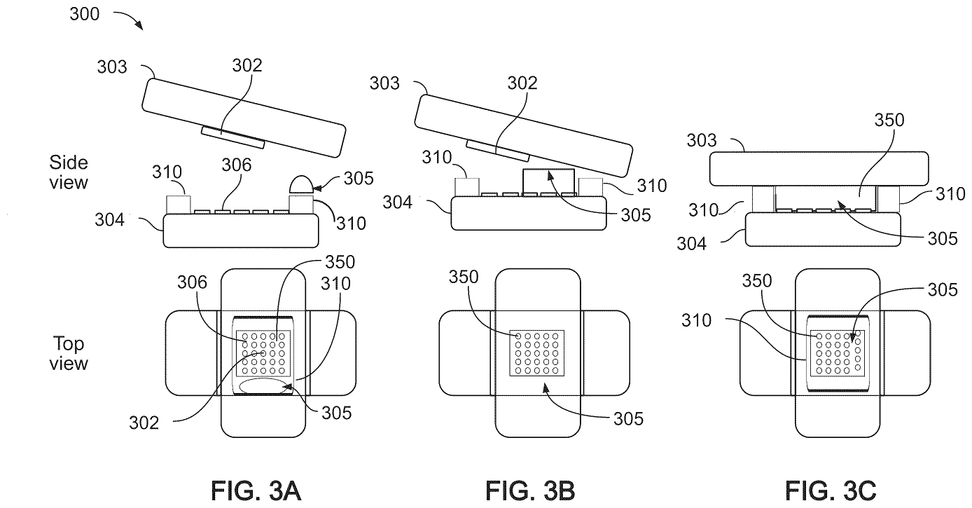

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

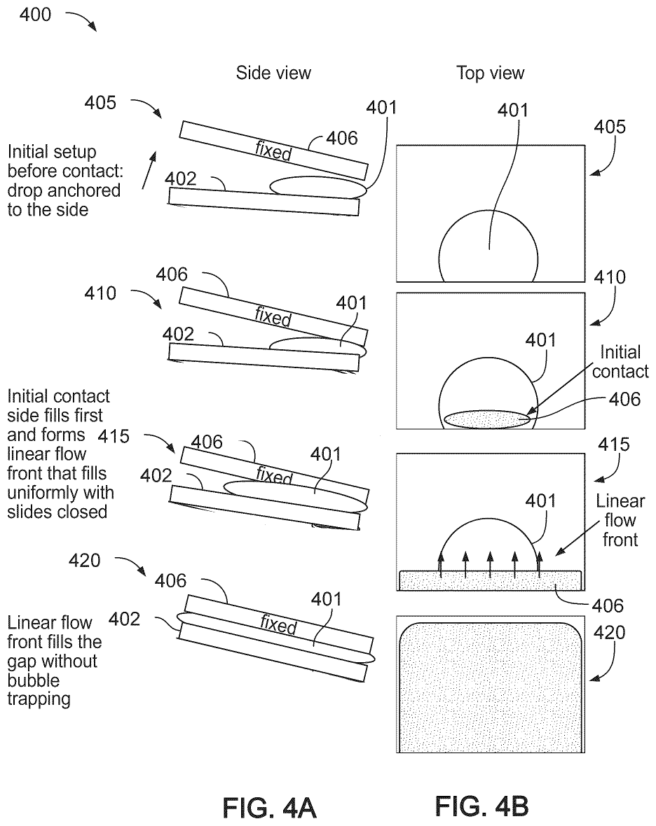

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663, which is herein incorporated by reference. Some quality control measures are described in Section (II)(h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of PCT Publication No. WO2020/176788 and/or U.S. Patent Application Publication No. 2020/0277663, which is herein incorporated by reference. Exemplary features and geometric attributes of an array can be found in Sections (II) (d)(i), (II)(d)(iii), and (II)(d)(iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of PCT Publication No. WO2020/176788 and/or U.S. Patent Application Publication No. 2020/0277663, which is herein incorporated by reference.

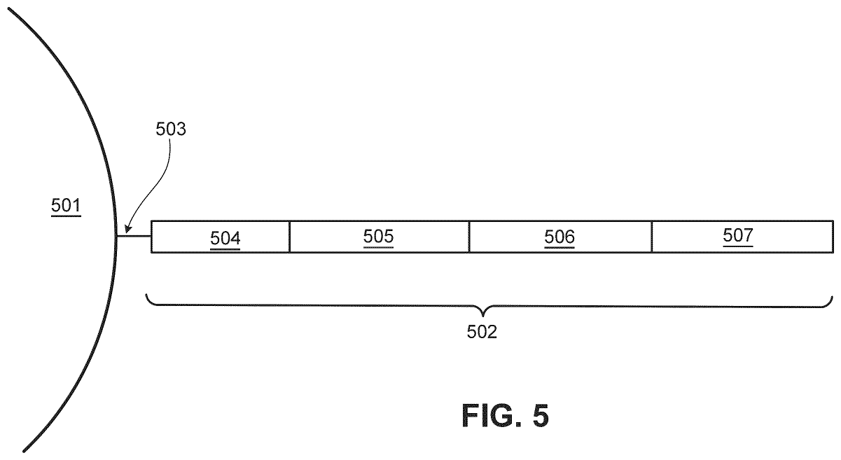

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

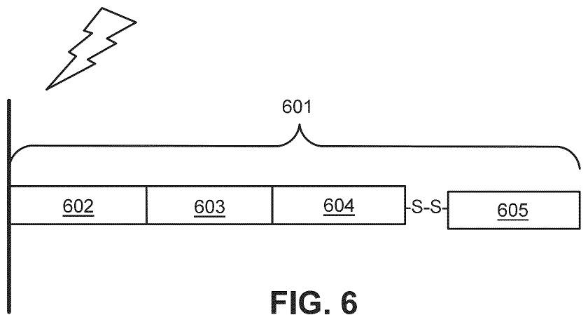

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

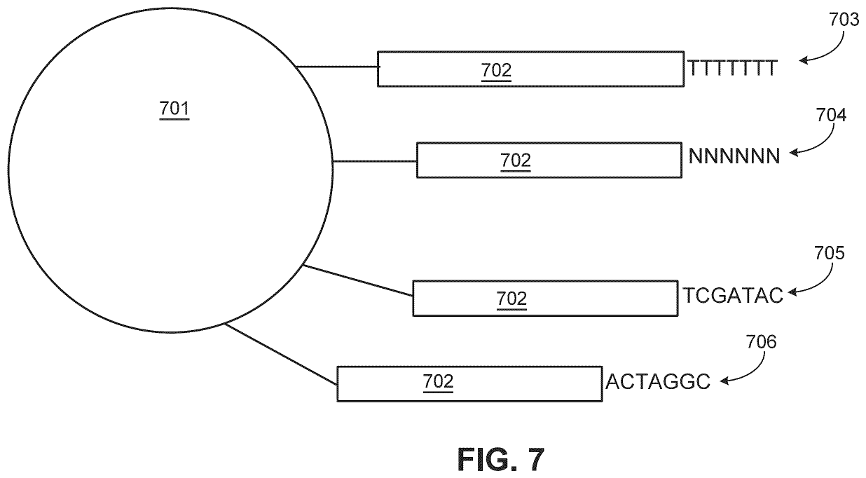

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

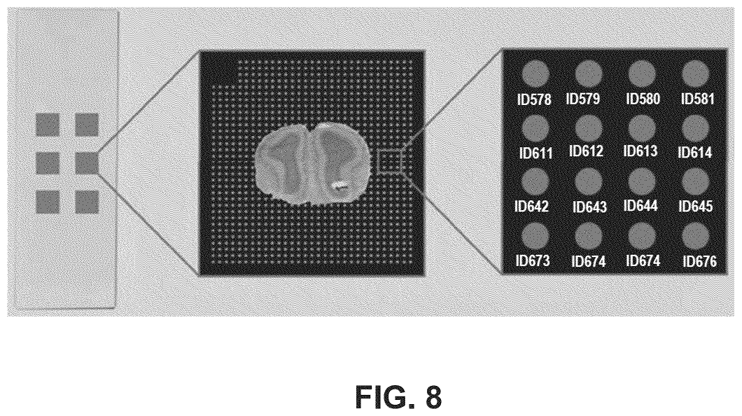

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14): e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

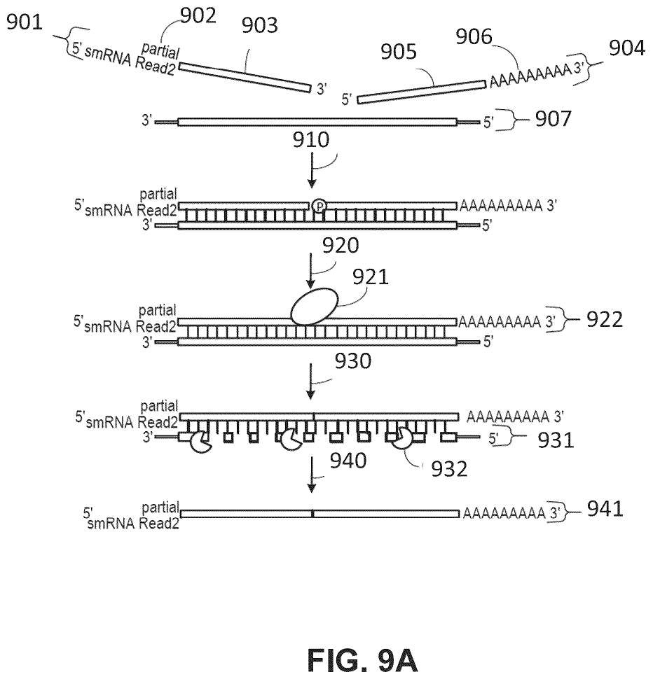

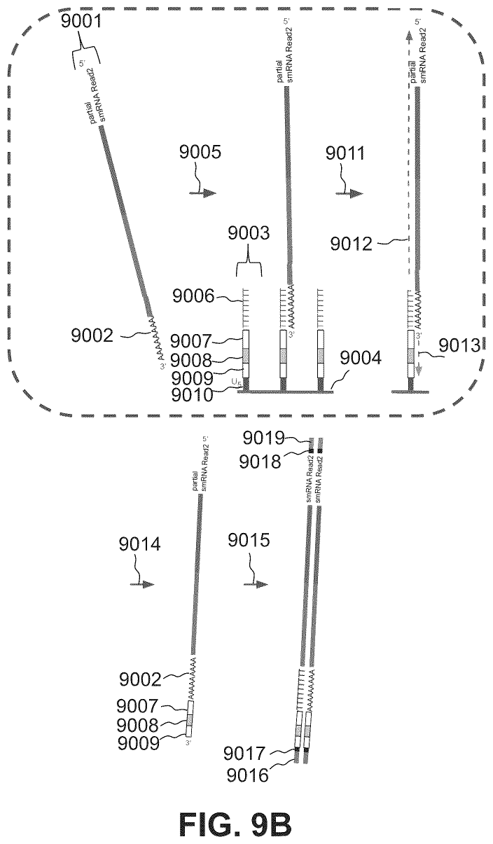

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Publication. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the polymerization reagents can be used to extend the capture probes 9011 to produce spatially-barcoded full-length cDNA 9012 and 9013 from the captured ligation products (e.g., ligation products). The ligation products can be extended using the capture probe as a template to include a complement of the capture probe, thereby generating extended ligation products.

In some embodiments, the extended ligation products can be denatured 9014, released from the capture probe, and transferred (e.g., to a clean tube) for amplification and/or library construction. The spatially-barcoded ligation products can be amplified 9015 via PCR prior to library construction. P5 9016, i5 9017, i7 9018, and P7 9019 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663, which is herein incorporated by reference.

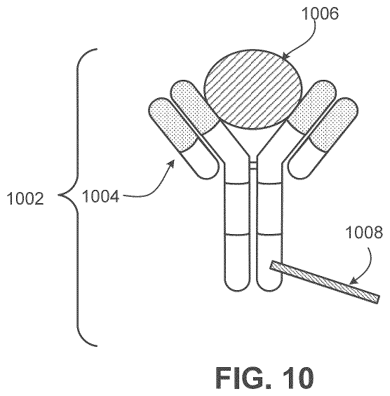

FIG. 10 is a schematic diagram of an exemplary analyte capture agent 1002 comprised of an analyte binding moiety 1004 and an analyte-binding moiety barcode domain 1008. The exemplary analyte binding moiety 1004 is capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment).

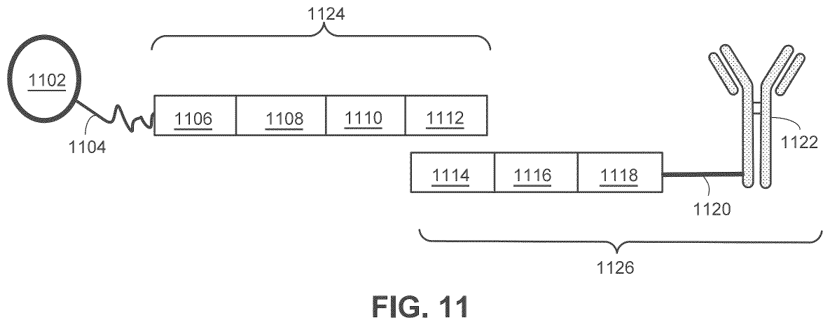

FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable, thermal-cleavable, or an enzyme cleavable linker. In some instances, the cleavable linker is a disulfide linker. A disulfide linker can be cleaved by use of a reducing agent, such as dithiothreitol (DTT), beta-mercaptoethanol (BME), or Tris(2-carboxyethyl)phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a sealable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Spatial Analysis of RNA Methylation Status

N⁶-methyladenosine (m⁶A) is the most abundant internal messenger RNA modification in higher eukaryotes and plays a role in regulating cellular processes. m⁶A constitutes a regulatory network extensively involved in physiological and pathological processes. It alters mRNA processing, structure, translation, and decay without changing the genetic code. The regulatory mechanisms governed by m⁶A are highly heterogeneous; functional outcomes of m⁶A modifications vary across different transcripts, different regions in the same transcript and different cell types.

Methylation is present in all types of RNA and in all kingdoms of life. However, the distribution of various methylation types and their abundance varies among Archea, Bacteria, and eukaryotes. Methyl groups are introduced by RNA methylating enzymes, which are mechanistically diverse and can be divided into a few broad groups based on the electronic demand of the substrate.

Examples of prokaryotic RNA editing enzymes include those in the tRNA-specific adenosine deaminase (tadA) family. See e.g., Wolf et al., EMBO J. 2002 Jul. 15; 21(14): 3841-3851, which is incorporated by reference in its entirety. TadA enzymes convert adenosine to inosine (I), which has been observed in viral transcripts and eukaryotic mRNAs. Because I is read as guanosine (G) by the translational machinery, RNA editing can change codon specificity and therefore the amino acid sequence of the encoded protein, resulting in multiple protein products with different biological function from a single mRNA precursor.

Nucleobase heteroatoms and ribose hydroxyl groups are methylated by enzymes utilizing an SN2 displacement mechanism between the nucleophilic heteroatom and electrophilic methyl group of S-adenosyl-L-methionine (SAM). Methylation of C5 carbon atoms of cytosine and uridine is accomplished by enzyme-mediated conjugate addition, which builds nucleophilic character at the substrate carbon. Unique among RNA methylating enzymes are those that methylate C2 and C8 carbons of adenosines via a distinctive radical mechanism. Alterations using RNA editing enzymes can lead to manifestations of diseases such as neurodegenerative diseases, autoimmune diseases, cancers, cardiomyopathies, and respiratory chain deficiencies. See Stojkovic et al., Curr Opin Chem Biol. 2017 December; 41:20-27, which is incorporated by reference in its entirety.

Provided herein are methods for identifying methylation status of RNA in a biological sample and determining the location of a methylated nucleic acid (e.g., RNA). “Methylation status” as used herein refers to identifying the presence of one or more methyl groups in an RNA analyte, e.g., identifying one or more methylated nucleosides. In some instances, the one or more methyl groups is present on one or more adenosines, e.g., N⁶-methyladenosine. In some instances, the disclosure features a method for identifying a methylation status of an RNA analyte in a biological sample, the method comprising: (a) contacting the biological sample with a first substrate; (b) permeabilizing the biological sample; (c) deaminating the RNA in the biological sample by converting one or more non-methylated adenosines in the RNA to inosines, thereby generating a deaminated RNA; (d) hybridizing the deaminated RNA to a capture domain of a capture probe affixed to an array, wherein the capture probe further comprises a spatial barcode; and (e) determining (i) the sequence of the spatial barcode, or the complement thereof, and (ii) all or part of the sequence of the deaminated RNA, or a complement thereof, and using the determined sequences of (i) and (ii) to identify the methylation status of the RNA in the biological sample.

In another instance, provided herein are methods for identifying methylation status of RNA in a biological sample, the method comprising: (a) contacting the biological sample with a first substrate; (b) permeabilizing the biological sample; (c) deaminating the RNA in the biological sample by converting one or more non-methylated adenosines in the RNA to inosines, thereby generating a deaminated RNA; (d) contacting the biological sample with a plurality of probes comprising a first probe and a second probe, wherein the first probe or the second probe hybridize to the deaminated RNA and wherein: the first probe or the second probe comprises a sequence complementary to a first sequence of the deaminated RNA; and the second probe comprises a sequence complementary to a capture domain of a capture probe affixed to an array, wherein the capture probe further comprises a spatial barcode; (e) ligating the first probe and the second probe, thereby generating a ligation product; (f) hybridizing the ligation product to the capture domain of the capture probe; and (g) determining (i) the sequence of the spatial barcode, or the complement thereof, and (ii) all or part of the sequence of the ligation product, or a complement thereof, and using the determined sequences of (i) and (ii) to identify the methylation status of the RNA in the biological sample.

Also provided herein are methods for determining the location of a methylated RNA molecule in a biological sample, comprising: (a) contacting the biological sample with an adenosine deaminase such that one or more non-methylated adenosines in the RNA molecule is converted to inosine, thereby generating a deaminated RNA molecule; (b) hybridizing the deaminated RNA molecule to a capture domain of a capture probe of an array comprising a plurality of capture probes, wherein the capture probe further comprises a spatial barcode; (c) extending the capture probe using the deaminated RNA molecule as a template, thereby generating an extended capture probe; and (d) determining sequences of (i) the spatial barcode, or a complement thereof, and (ii) all or part of the extended capture probe, or a complement thereof, and using the determined sequences of (i) and (ii) to determine the location of the methylated RNA molecule in the biological sample.

In another instance, provided herein are methods for determining the location of a methylated RNA molecule in a biological sample, the method comprising: (a) providing the biological sample mounted on a first substrate; (b) contacting the biological sample with an adenosine deaminase such that one or more non-methylated adenosines in the RNA molecule is converted to inosine, thereby generating a deaminated RNA molecule; (c) contacting the biological sample with a plurality of probes comprising a first probe and a second probe, wherein the first probe or the second probe hybridize to the deaminated RNA molecule and wherein: the first probe and the second probe comprise a sequence complementary to a first sequence of the deaminated RNA molecule, wherein the first sequence of the deaminated RNA molecule comprises inosine and/or adenosine; and the second probe comprises a sequence complementary to a capture domain of a capture probe of an array comprising a plurality of capture probes, wherein the capture probe further comprises a spatial barcode; (d) ligating the first probe and the second probe, thereby generating a ligation product; (e) hybridizing the ligation product to the capture domain of the capture probe; and (f) determining sequences of (i) the spatial barcode, or a complement thereof, and (ii) all or part of the ligation product, or a complement thereof, and using the determined sequences of (i) and (ii) to determine the location of the methylated RNA molecule in the biological sample.

In another instance, provided herein are methods for determining the location of a methylated RNA molecule in a biological sample using in situ methods, e.g., using padlock probes. For instance, a padlock probe can hybridize to an analyte (e.g., an RNA molecule), a ligation product, or an extended capture probe. In some embodiments, the methods disclosed herein include (a) providing the biological sample mounted on a first substrate; (b) contacting the biological sample with an adenosine deaminase such that one or more non-methylated adenosines in the RNA molecule is converted to inosine, thereby generating a deaminated RNA molecule; (c) contacting the biological sample with a plurality of probes comprising a first probe and a second probe, wherein the first probe or the second probe hybridize to the deaminated RNA molecule and wherein: the first probe and the second probe comprise a sequence complementary to a first sequence of the deaminated RNA molecule, wherein the first sequence of the deaminated RNA molecule comprises inosine and/or adenosine; (d) ligating the first probe and the second probe, thereby generating a ligation product; (e) hybridizing a padlock probe to the ligation product; (f) circularizing the padlock probe using e.g., a ligase; (g) optionally amplifying the padlock probe, thereby generating an amplified padlock probe; (h) detecting the amplified padlock probe using one or more detection probes, optionally wherein the one or more detection probes collectively comprise one or more fluorophores.

In another instance, a padlock probe can hybridize to a deaminated analyte (e.g., a deaminated RNA molecule). In some embodiments, the methods disclosed herein include (a) providing the biological sample mounted on a first substrate; (b) contacting the biological sample with an adenosine deaminase such that one or more non-methylated adenosines in the RNA molecule is converted to inosine, thereby generating a deaminated RNA molecule; (c) contacting the biological sample with a padlock probe, wherein the padlock probe hybridizes to the deaminated RNA molecule, (d) circularizing the padlock probe using, e.g., a ligase; (e) optionally amplifying the padlock probe, thereby generating an amplified padlock probe; and (f) detecting the amplified padlock probe using one or more detection probes, optionally wherein the one or more detection probes collectively comprise one or more fluorophores.

In some embodiments, the methods described herein include a step of permeabilizing the biological sample. In some instances, the permeabilization step occurs prior to deaminating the analyte in the sample. In some instances, permeabilizing includes contacting the biological sample with a permeabilization reagent. Any suitable permeabilization reagent described herein can be used. In some embodiments, the permeabilization reagent is an endopeptidase. Endopeptidases that can be used include, but are not limited to, trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin.

In some embodiments, the methods described herein include deaminating the analyte (e.g., RNA molecule). In some embodiments, the deaminating is achieved by contacting the biological sample or the analyte with one or more enzymatic or chemical deaminating agent(s). Any suitable deaminating agents can be used in the methods described herein. In some preferred embodiments, the deaminating agent is an adenosine deaminase. In some embodiments, the deaminating agent is a TadA deaminase or variant or functional equivalent thereof. In some embodiments, the deaminating agent is a tRNA-specific adenosine deaminase (TadA) from E. coli, i.e., an ecTadA or variant or functional equivalent thereof. In some embodiments, the adenosine deaminase is TadA8.20.

In some embodiments, the deaminating step includes contacting the biological sample with a composition comprising the deaminating agent.

In some instances, the deaminating step is performed in the presence of the capture probes on the array. In some instances, the capture probes include one or more methylated adenosines that are not deaminated during the deamination step.

In some embodiments, unmethylated adenosines, if present in the RNA analyte in the sample, are deaminated into inosines. In some embodiments of using first and second probes, after the hybridizing of the first and second probes with the deaminated analyte, the first or second probe is extended wherein each inosine, if present in the analyte, is base-paired with a cytosine. In some embodiments, the extended first or second probe comprises a cytosine in each position corresponding to the unmethylated adenosines in the analyte in the sample. In some embodiments, all of the unmethylated adenosines in the RNA analyte are converted to inosines by the deaminating step.

After deamination, in some instances, one or more probes are added to the biological sample. In some instances, the one or more probes is at least 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99. 100, at least about 105, at least about 110, at least about 115, at least about 120, at least about 125, at least about 135, at least about 140, or more nucleotides in length. In some embodiments, the probe includes sequences that are complementary or substantially complementary to an RNA analyte. By substantially complementary, it is meant that the probe is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in an analyte. In some instances, the first probe and the second probe o hybridize to adjacent sequences on an analyte.

In some instances, the probes used herein are designed to hybridize to sequences that are mutated (e.g., from adenosine to inosine) as a result of the deamination step. In some instances, the probes used herein are designed to hybridize to sequences that are not mutated (e.g., adenosines that are methylated are not mutated during the deamination step) as a result of the deamination step. In some instances, the probes used herein are designed to hybridize to sequences that include at least one nucleotide that is mutated as a result of the deamination step. In some instances, the probes used herein are designed to hybridize to sequences that include more than one (e.g., 2, 3, 4, 5, or more) nucleotide that is mutated as a result of the deamination step.

In some instances, the one or more probes further includes a sequence that can hybridize to a capture probe sequence (e.g., on an array, as described herein). In some instances, the sequence that hybridizes to the capture probe is a poly-adenylation (poly(A)) sequence. In some instances, the sequence that hybridizes to the capture probe is a degenerate (e.g., random) sequence. In some instances, the sequence that hybridizes to the capture probe is designed to be a specific sequence.

In some instances, only one probe hybridizes to a target RNA molecule. In some instances, the probe hybridizes to a sequence that has been affected (e.g. includes a mutation) after deamination. In some instances, the mutation is an adenosine to an inosine. In some instances, the analyte includes more than one mutation as a result of the deamination step. In some instances, there are 1, 2, 3, 4, 5, 6, 7, 8, 9, 10 or more adenosines that are mutated to an inosine (i.e., the adenosines are not methylated). In some instances, there are 1, 2, 3, 4, 5, 6, 7, 8, 9, 10 or more adenosines that are not mutated to an inosine (i.e., the adenosines are methylated). In some instances, the probe is designed to detect one or more deamination mutations.

In some instances, more than one (e.g., 2, 3, 4, 5, or more) probe hybridizes to a target RNA molecule. In some instances, two probes hybridize to sequences that are adjacent to one another. In some instances, because the probes hybridize to adjacent sequences, ligation can occur between the adjacent probes after hybridization (i.e., no extension step as described herein is required). In some instances, two probes hybridize to sequences of an analyte that are separated by at least 1, 2, 3, 4, 5, 6, 7, 8, 9, 10, about 15, about 20, about 25, about 30, about 35, about 40, about 45, about 50, about 55, about 60, about 65, about 70, about 75, about 80, about 85, about 90, about 95, about 100, about 125, about 150, about 175, about 200, about 250, about 300, about 350, about 400, about 450, about 500, about 600, about 700, about 800, about 900, or about 1000 nucleotides. In some instances, the probes hybridize to a sequence that has been modified (e.g. includes a mutation) after deamination. In some instances, the mutation is an adenosine to an inosine. In some instances, the analyte includes more than one mutation as a result of the deamination step. In some instances, there are 1, 2, 3, 4, 5, 6, 7, 8, 9, 10 or more adenosines that are mutated to an inosine (i.e., the adenosines are not methylated). In some instances, there are 1, 2, 3, 4, 5, 6, 7, 8, 9, 10 or more adenosines that are not mutated to an inosine (i.e., the adenosines are methylated). In some instances, the probes are designed to detect one or more deamination mutations.

In some instances, the sequence between the two regions of an analyte where the probes hybridize (i.e., the intervening sequence) include one or more nucleotides that is mutated (e.g., from adenosine to inosine) during the deamination step. In some instances, the intervening sequence includes at least 2, 3, 4, 5, 6, 7, 8, 9, 10, or more nucleotides that have been mutated during the deamination step described herein.

The one or more probes include a sequence that specifically binds to a region on the target RNA analyte. In some embodiments, at least one of the probes is extended using a polymerase. In some instances, one of the probes is extended to fill in the gap between the two hybridized probes.

In some embodiments, the capture probe is extended using a polymerase.

In some embodiments, the polymerase is a DNA polymerase. In some embodiments, the DNA polymerase is a thermostable DNA polymerase including, but are not limited to, DNA polymerase such as Phusion, Hot Start Taq DNA Polymerase, and EpiMark® Hot Start Taq DNA Polymerase.

The extended capture probe can be amplified using any suitable amplifying methods described herein. In some embodiments, the amplifying is isothermal. In some embodiments, the amplifying can be performed using a thermocycling protocol (e.g., lid temperature and pre-equilibrate at about 95° C., denaturing at about 95° C. for about 1 min, reannealing at about 60° C. for about 60 seconds, extension at about 72° C. for about 2 minutes, repeating the denaturing, reannealing, and extension about 15-30 times, and then held at about 4° C.). The reaction mixture further includes all necessary polymerases and buffers. In some embodiments, the polymerase can be a DNA polymerase. In some embodiments, the DNA polymerase is a thermostable DNA polymerase including, but not limited to, Phusion, Hot Start Taq DNA Polymerase, and EpiMark® Hot Start Taq DNA Polymerase.

Any suitable ligase can be used in the methods described herein. In some instances, the probes may be subjected to an enzymatic ligation reaction, using a ligase (e.g., T4 RNA ligase 2, a splintR ligase, a single stranded DNA ligase). In some instances, the ligase is T4 DNA ligase. In some instances, the ligase is T4 RNA ligase 2, also known as Rnl2, which ligates the 3′ hydroxyl end of an RNA to the 5′ phosphate of DNA in a double stranded structure. T4 DNA ligase is an enzyme belonging to the DNA ligase family of enzymes that catalyzes the formation of a covalent phosphodiester bond from a free 3′ hydroxyl group on one DNA molecule and a free 5′ phosphate group of a second, separate DNA molecule, thus covalently linking the two DNA strands together to form a single DNA strand. In some instances, the ligase is splintR ligase. SplintR Ligase, also known as PBCV-1 DNA Ligase or Chorella virus DNA Ligase, efficiently catalyzes the ligation of adjacent, single-stranded DNA oligonucleotides splinted by a complementary RNA strand. In some instances, the ligase is a single-stranded DNA ligase. In some embodiments, the ligase is a pre-activated T4 DNA ligase. Methods of utilizing a pre-activated T4 DNA ligase are further disclosed in U.S. Publication No. 2010-0184618-A1, which is incorporated by reference in its entirety.

In some embodiments, adenosine triphosphate (ATP) is added during the ligation reaction. DNA ligase-catalyzed sealing of nicked DNA substrates is first activated through ATP hydrolysis, resulting in covalent addition of an AMP group to the enzyme. After binding to a nicked site in a DNA duplex, the ligase transfers this AMP to the phosphorylated 5′-end at the nick, forming a 5′-5′ pyrophosphate bond. Finally, the ligase catalyzes an attack on this pyrophosphate bond by the OH group at the 3′-end of the nick, thereby sealing it, whereafter ligase and AMP are released. If the ligase detaches from the substrate before the 3′ attack, e.g. because of premature AMP reloading of the enzyme, then the 5′ AMP is left at the 5′-end, blocking further ligation attempts. In some instances, ATP is added at a concentration of about 1 μM, about 10 μM, about 100 μM, about 1000 μM, or about 10000 μM during the ligation reaction.

In some instances, cofactors that aid in joining of the probes are added during the ligation process. In some instances, the cofactors include magnesium ions (Mg2+). In some instances, the cofactors include manganese ions (Mn2+). In some instances, Mg2+is added in the form of MgCl2. In some instances, Mn2+ is added in the form of MnCl2. In some instances, the concentration of MgCl2 is at about 1 mM, at about 10 mM, at about 100 mM, or at about 1000 mM. In some instances, the concentration of MnCl2 is at about 1 mM, at about 10 mM, at about 100 mM, or at about 1000 mM.

In some instances, the ligation reaction occurs at a pH in the range of about 6.5 to 9.0, of about 6.5 to 8.0, of about 7.5 to 8.0, of about 7.5, or of about 8.0.

In some embodiments, the methods described herein further comprise releasing the ligated probe from the analyte. In some embodiments, the releasing occurs before hybridizing the ligated probe to the capture probe. In some embodiments, the releasing comprises contacting the ligated probe with an endoribonuclease. Any suitable endoribonuclease can be used in the methods described herein. In some instances, the ligated probe is dehybridized from the analyte by heating the sample. In some instances, the extended probe is dehybridized at about 50° C., about 55° C., about 60° C., about 65° C., about 70° C., or about 75° C. In some instances, the ligated probe is dehybridized from the analyte electrochemically.

In some embodiments, methods provided herein include contacting a biological sample with a substrate, wherein the capture probe is affixed to the substrate (e.g., immobilized to the substrate, directly or indirectly). In some embodiments, the capture probe includes a spatial barcode and a capture domain. In some embodiments, the capture probe binding domain of the ligated probe specifically binds to the capture domain. After hybridization of the ligated probe to the capture probe, the ligated probe is extended at the 3′ end to make a copy of the additional components (e.g., the spatial barcode) of the capture probe. In some embodiments, methods of ligated probe capture as provided herein include permeabilization of the biological sample such that the capture probe can more easily hybridize to the captured ligated probe (i.e., compared to no permeabilization).

In some embodiments, the methods described herein comprise hybridizing the ligated probe to a capture probe on an array. In some instances, an array includes a plurality of capture probes. In some instances, each capture probe of the plurality comprises a sequence that is substantially complementary to at least a portion of the analyte or a complement thereof, or a capture probe binding domain of the ligated probe. In some instances, each capture probe includes a poly-thymine sequence that hybridizes to a poly-adenylation sequence e.g., in one or more of the probes or analyte. In some embodiments, the capture probe further includes a UMI, or a complement thereof. In some embodiments, the capture probe includes a spatial barcode.

In some instances, after the ligated probe hybridizes to the capture probe, the method disclosed herein includes extending the capture probe using the extended probe as a template; thereby generating an extended capture probe. In some instances, the capture probe is extended wherein each inosine, if present in the extended probe, is base-paired with a cytosine. In some embodiments, the extended probe comprises a guanosine in each position corresponding to the unmethylated adenosine in the RNA analyte in the biological sample. In some embodiments, the methylated adenosine, e.g., m⁶A, if present in the analyte in the sample, remains unchanged.

In some embodiments, reverse transcription (RT) reagents can be added to biological samples. Incubation with the RT reagents can produce spatially-barcoded cDNA from the captured analytes (e.g., RNA nucleic acids that are or are not methylated). Second strand reagents (e.g., second strand primers, enzymes) can be added to the biological sample on the slide to initiate second strand DNA synthesis.

The resulting DNA can be denatured from the capture probe template and transferred (e.g., to a clean tube) for amplification, and/or library construction as described herein. The spatially-barcoded DNA can be amplified via PCR prior to library construction. The DNA can then be enzymatically fragmented and size-selected in order to optimize the DNA amplicon size. Sequencing specific nucleic acid sequences such as P5, P7, sample indices such as i7, and i5 and sequencing primer sequences such as TruSeq Read 2 (exemplary sequences that are used in Illumina NGS sequencing workflows) can be added via End Repair, A-tailing, Adaptor Ligation, and PCR. The DNA fragments can then be sequenced using paired-end sequencing using TruSeq Read 1 and TruSeq Read 2 as sequencing primer sites. In some instances, the DNA library is sequenced using any method described herein. In some instances, the sequence of the DNA is determined via sequencing. In some instances, the sequencing is long read sequencing without, for example, single molecule real time sequencing (SMRT) or nanopore sequencing. In some instances, the spatial barcode is sequenced, providing the location of the analyte.

In some embodiments, the method further comprises washing the biological sample. In some instances, a wash step occurs between the deamination step and the step where the probes are added. In some embodiments, the washing step is conducted to remove all or a part of the deaminating agent. In some embodiments, the washing step does not remove the RNA analyte in the biological sample. In some embodiments, the washing step removes an insignificant amount of the analyte in the biological sample. In some instances, a wash step occurs after hybridizing the oligonucleotides. In some instances, this wash step removes any unbound oligonucleotides and can be performed using any technique or solution disclosed herein or known in the art. In some embodiments, multiple wash steps are performed to remove unbound oligonucleotides.

In some instances, detection of an analyte or analyte-derived molecule (e.g., a ligation product) can be achieved by in situ methods. For instance, after hybridization of the analyte or analyte-derived molecule, a padlock probe can be added to the biological sample where it hybridizes to the analyte or analyte-derived molecule. After hybridization, the padlock probe can be ligated and amplified via rolling circle amplification (RCA). Initially, in some instances, a padlock probe hybridizes to an analyte or analyte derived molecule, or the complement thereof. As used herein, a “padlock probe” refers to an oligonucleotide that includes, at its 5′ and 3′ ends, sequences (e.g., a first sequence at the 5′ end and a second sequence at the 3′ end) that are complementary to adjacent or nearby portions (e.g., a first portion and a second portion) of the analyte or an analyte derived molecule. Padlock probes are designed such that they comprise multiple substantially or fully complementary sequences to the analyte or analyte derived molecule. As described herein, an analyte includes nucleic acids (e.g., DNA or RNA) in some instances. An example of an analyte derived molecule includes a molecule comprising a protein binding moiety conjugated to an oligonucleotide. A further example is a product of RNA-templated ligation (RTL) as disclosed in US 2021/0348221, US 2021/0285046, and WO 2021/133849, each of which is incorporated by reference in its entirety. Examples and description of analyte derived molecule (also referred to as analyte binding moieties) has been described in WO 2020/176788 and/or U.S. 2020/0277663, each of which is incorporated by reference.

In some instances, Specific Nucleic Acid detection via Intramolecular Ligation (snail) probes are used. SNAIL-RCA (e.g., RCA using snail probes) uses an adaptation of the padlock probe/proximity ligation/rolling circle amplification process and has been shown to amplify the signal from RNA present at relatively low levels of expression. See e.g., O'Huallachain, M. et al. Communications Biology volume 3, Article number: 213 (2020); Nilsson, M. et al. Science. 7522346 (1994); and Wang, X. et al. Science. aat5691 (2018); each of which is incorporated by reference in its entirety. SNAIL-RCA was adopted for directed amplification of RNA in cells by proximity ligation. Typically, two primers are used, one of which binds to its target (e.g., an extended capture probe; a ligation product; or an oligonucleotide from an analyte capture agent; or any complements thereof) at a 3′ position and contains the sequence cognate to a common ligation junction. The second primer contains a sequence proximal, which binds slightly upstream on the target, but which also anneals to the ligation junction. After ligation of the two SNAIL primers, rolling circle amplification occurs which results in amplification of the target or complement thereof.

Upon hybridization of the padlock probe or snail probe to the first and second portions (i.e., sequences) of the analyte or analyte derived molecule, the two ends of the padlock probe are either brought into proximity, or an end is extended until the two ends are brought into proximity, allowing circularization of the padlock probe or snail probe, e.g., by ligation (e.g., ligation using any of the methods described herein). The product generated by ligation of the padlock probe or the snail probe can be referred to as the “circularized padlock probe” or “circularized snail probe.” After circularization of the padlock probe or snail probe, rolling circle amplification can be used to amplify the circularized padlock probe or snail probe.

In some embodiments, a first sequence of a padlock probe or snail probe includes a sequence that is substantially complementary to a first portion of the analyte or analyte derived molecule. In some embodiments, the first portion of the analyte or analyte derived molecule is 5′ to the second portion of the analyte or the analyte derived molecule. In some embodiments, the first sequence is at least 70% identical (e.g., at least 75% identical, at least 80% identical, at least 85% identical, at least 90% identical, at least 95% identical, or at least 99% identical) to the first portion.

In some embodiments, a backbone sequence of a padlock probe or snail probe includes a sequence that is substantially complementary to an amplification primer. The amplification primer can be a primer used in a rolling circle amplification reaction (RCA) of the ligated padlock probe or snail probe hybridized to the analyte or analyte derived molecules. Rolling circle amplification is well known in the art and includes a process by which circularized nucleic acid molecules are amplified with a DNA polymerase with strand displacement capabilities (and other necessary reagents for amplification to occur), thereby creating multiple concatenated copies of the circularized nucleic acid molecules. In some embodiments, the backbone sequence includes a functional sequence. In some embodiments, the backbone sequence includes a unique molecule identifier (UMI) or barcode sequence (e.g., any of the exemplary barcode sequences described herein). In some embodiments, the barcode sequence includes a sequence that is substantially complementary to an amplification primer. In some instances, the backbone UMI or backbone barcode sequence comprises a sequence (e.g., n=5-50 nucleotides) that is specific to the padlock probe or snail probe. In some instances, the backbone UMI or backbone barcode sequence in the padlock probe or snail probe can be used to identify the padlock or snail sequence.

In some embodiments, a second sequence of a padlock probe or snail probe includes a sequence that is substantially complementary to a second portion of the analyte or analyte derived molecule. In some embodiments, the second portion of the analyte or analyte derived molecule is 3′ to the first portion of the analyte or the analyte derived molecule. In some embodiments, the second sequence is at least 70% identical (e.g., at least 75% identical, at least 80% identical, at least 85% identical, at least 90% identical, at least 95% identical, or at least 99% identical) to the second portion.

In some embodiments, the first sequence is substantially complementary to a first portion of the analyte or analyte derived molecule that is directly adjacent to the second portion of the analyte or analyte derived molecule to which the second sequence is substantially complementary. In such cases, the first sequence is ligated to the second sequence, thereby creating a circularized padlock probe or snail probe.

In some embodiments, the first sequence is substantially complementary to a first portion of the analyte or analyte derived molecule that is not directly adjacent to the second portion of the analyte or analyte derived molecule to which the second sequence is substantially complementary. In such cases, a “gap” exists between where the first sequence is hybridized to the first potion and where the second sequence is hybridized to the second portion. In some embodiments, there is a nucleotide sequence (e.g., a gap) in the analyte or analyte derived molecule between the first portion and the second portion of at least 1-100, 1-90, 1-80, 1-70, 1-60, 1-50, 1-40, 1-30, 1-20, 1-10, 1-9, 1-8, 1-7, 1-6, 1-5, 1-4, 1-3, 1-2 or 1 nucleotide(s). In a non-limiting example, a first sequence having a sequence that is complementary to a sequence 5′ of the gap and a second sequence having a sequence that is complementary to a sequence 3′ of the gap each bind to an analyte leaving a sequence (e.g., the “gap”) in between the first and second sequences that is gap-filled thereby enabling ligation and generation of the circularized padlock probe or snail probe. In some instances, to generate a padlock probe or snail probe that includes a first sequence and a second sequence that are close enough to one another to initiate a ligation step, the second sequence is extended enzymatically (e.g., using a reverse transcriptase). In some embodiments, the “gap” sequence between the first sequence and the second sequence include one nucleotide, two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, at least 25 nucleotides, at least 30 nucleotide, at least 35 nucleotides, at least 40 nucleotides, at least 45 nucleotides, or at least 50 nucleotides.

In some embodiments, extending the second sequence of the padlock probe or snail probe includes a nucleic acid extension reaction (e.g., any of the nucleic acid extension reactions described herein). In some embodiments, extending the second sequence of the padlock probe or snail probe includes reverse transcribing the analyte or analyte derived molecule. In some embodiments, extending the second sequence of the padlock probe or snail probe includes using a reverse transcriptase (e.g., any of the reverse transcriptases described herein). In some embodiments, extending the second sequence of the padlock probe or snail probe includes using a Moloney Murine Leukemia Virus (M-MMLV) reverse transcriptase. In some embodiments, the reverse transcriptase includes strand displacement properties. In some embodiments, extending the second sequence of the padlock probe generates a sequence that is complementary to the analyte or the analyte derived molecule. In some embodiments, extending the second sequence of the padlock probe or snail probe generates an extended second sequence of the padlock probe or snail probe that is complementary to the analyte or analyte derived molecule. In some embodiments, second sequence of the padlock probe or snail probe generates a sequence that is adjacent to the first sequence of the padlock probe or snail probe.

In some embodiments, the ligation step includes ligating the second sequence to the first sequence of the padlock probe or snail probe using enzymatic or chemical ligation. In some embodiments where the ligation is chemical ligation, the ligation reaction comprises click chemistry. In some embodiments where the ligation is enzymatic, the ligase is selected from a T4 RNA ligase (Rnl2), a SplintR ligase, a single stranded DNA ligase, or a T4 DNA ligase. In some embodiments, the ligase is a T4 RNA ligase (Rnl2) ligase. In some embodiments, the ligase is a pre-activated T4 DNA ligase as described herein. A non-limiting example describing methods of generating and using pre-activated T4 DNA include U.S. Pat. No. 8,790,873, the entire contents of which are herein incorporated by reference.

This disclosure includes amplification of a circularized padlock probe or snail probe. In a non-limiting example, the method includes an amplifying step where one or more amplification primers are hybridized to the circularized padlock probe or snail probe and the circularized padlock probe or snail probe is amplified using a polymerase. Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes. The amplification product(s) can be detected by hybridization of detection probes to the amplification product, which identify the location of the analyte in the biological sample. The location of the analyte can then be compared against an initial (or original) location of an analyte based on imaging prior to permeabilization of the biological sample and migration of the analytes. In some embodiments, the amplifying step includes rolling circle amplification (RCA).

As used herein, rolling circle amplification (RCA) refers to a polymerization reaction carried out using a single-stranded circular DNA (e.g., a circularized padlock probe or snail probe) as a template and an amplification primer that is substantially complementary to the single-stranded circular DNA (e.g., the circularized padlock probe or snail probe) to synthesize multiple continuous single-stranded copies of the template DNA (e.g., the circularized padlock probe or snail probe). In some embodiments, RCA includes hybridizing one or more amplification primers to the circularized padlock probe or snail probe and amplifying the circularized padlock probe or snail probe using a DNA polymerase with strand displacement activity, for example Phi29 DNA polymerase. In some embodiments, a first RCA reaction includes a first padlock probe or snail probe and a first amplification primer (or plurality of first amplification primers). A first RCA reaction can include the first padlock probe or snail probe hybridizing to a first analyte or first analyte derived molecule.

In some embodiments, an RCA reaction is carried out using a DNA polymerase and a dNTP mix including uracil, adenine, guanine and cytosine. In such cases, the uracils are incorporated into the amplified padlock probe or snail probe. In some embodiments, an RCA reaction is carried out using a DNA polymerase and a dNTP mix including uracil, adenine, guanine, cytosine and thymine. In such cases, uracils and thymines are both incorporated into the amplified padlock probe or snail probe. In some instances, the dNTP molecule is labeled (e.g., dATP, dCTP, dGTP, dUTP, and dTTP. The fluorescent label can be any fluorescent label known in the art, including but not limited to cyanine 5 dye (Cy-5), cyanine 3 dye (Cy-3), fluorescein, rhodamine, phosphor, coumarin, polymethadine dye, fluorescent phosphoramidite, Texas red, green fluorescent protein, acridine, cyanine, and 5-(2′-aminoethyl)-aminonaphthalene-1-sulfonic acid (EDANS).

This disclosure further includes methods for detection of a signal that corresponds to an amplified circularized padlock probe or snail probe (which thereby corresponds to an analyte or analyte binding molecule). In some embodiments, the method includes a step of detecting a signal corresponding to the amplified circularized padlock probe or snail probe, thereby identifying the location of the analyte in the biological sample. In some embodiments, the method includes a detecting step that includes determining (i) all or a part of the sequence of the amplified circularized padlock probe or snail probe on the substrate and using the determined sequence of the padlock probe or snail probe to identify the location of the analyte in the biological sample.

In some embodiments, the detecting step includes contacting the amplified circularized padlock probe or snail probe with a plurality of detection probes. In some embodiments, a detection probe of the plurality of detection probes includes a sequence that is substantially complementary to a sequence of the padlock probe or snail probe, circularized padlock probe or snail probe, or amplified circularized padlock probe or snail probe, and a detectable label. For example, the detection probe of the plurality of detection probes can include a sequence that is substantially complementary to a sequence of amplified circularized padlock probe or snail probe and a detectable label. By substantially complementary, it is meant that the detection probe is at least 70%, at least 75%, at least 80%, at least 85%, at least 90%, at least 91%, at least 92%, at least 93%, at least 94%, at least 95%, at least 96%, at least 97%, at least 98%, at least 99% complementary to a sequence in the amplified circularized padlock probe or snail probe. In some embodiments, the detectable label is a fluorophore.

In some embodiments, the detecting step includes contacting the amplified circularized padlock probe or snail probe with a detectable label that can hybridize to nucleic acid sequences in a non-sequence dependent manner. In such cases, the single-stranded copies of the template DNA produced by RCA can be detected using fluorescent dyes that non-specifically bind to the single-stranded nucleic acid (e.g., the amplified circularized padlock probe or snail probe). For example, in the case of an amplified circularized padlock probe or snail probe, a fluorescent dye can bind, either directly or indirectly, to the single stranded nucleic acid of the RCA product. The dye can then be detected as a signal corresponding to the amplified circularized padlock probe or snail probe (i.e., the location and/or the abundance of the circularized padlock probe or snail probe). Non-limiting examples of fluorescent dyes that can bind to single stranded nucleic acids include: TOTO®-1/TO-PRO®-1, TOTO®-3/TO-PRO®-3, TO-PRO®-5, Ethidium bromide, Ethidium homodimer-1 (EthD-1), YOYO®-1/YO-PRO®-1, YOYO®-3/YO-PRO®-3, 7-AAD (7-Aminoactinomycin D), and OliGreen®.

In some embodiments, the detecting step includes contacting the amplified circularized padlock probe or snail probe with a detection probe (e.g., any of the exemplary detection probes described herein (e.g., a detection probe that includes one or more fluorophores)) and a fluorescent dye that can label single-stranded DNA in a non-sequence dependent manner (e.g., any of the exemplary fluorescent dyes described herein). In such cases, the fluorescent dye can be used to normalize the fluorescent signal detected from the detection probe.

In some embodiments, detecting the signal or signals that correspond to the amplified circularized padlock probes or snail probes on the substrate include obtaining an image corresponding to the analyte and/or analyte derived molecule on the substrate. In some embodiments, the method further includes registering image coordinates to a fiducial marker.

In some embodiments, determining (i) all or a part of the sequence of the amplified circularized padlock probe or snail probe includes determining the barcode sequence on the padlock probe or snail probe, wherein the location of the analyte in the biological sample can be identified. In situ sequencing of the analyte or analyte derived molecule, the nucleic acid molecule, the analyte binding moiety barcode, or the ligation product process is described in PCT Patent Application Publication No. WO 2020/047005, which is incorporated by reference in its entirety.

The biological sample as used herein can be any suitable biological sample described herein or known in the art. In some embodiments, the biological sample is a tissue sample. In some embodiments, the biological sample is a tissue section. In some embodiments, the tissue is flash-frozen and sectioned. Any suitable methods described herein or known in the art can be used to flash-freeze and section the tissue sample. In some embodiments, the biological sample, e.g., the tissue sample, is flash-frozen using liquid nitrogen before sectioning. In some embodiments, the sectioning is performed using cryosectioning. In some embodiments, the methods further comprises a thawing step, after the cryosectioning. In some embodiments, the biological sample, e.g., the tissue sample is fixed, for example in methanol, acetone, PFA or is formalin-fixed and paraffin-embedded (FFPE). In some embodiments, wherein the biological sample is a FFPE tissue section, the method further includes decrosslinking the tissue section, and after enzymatic deamination, incorporating a poly(A) sequence at a 3′ end of the deaminated RNA.

The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to deaminating the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some embodiments, the tissue sample is obtained from a location where the RNA is differentially methylated compared to a reference location. The location where the RNA is differentially methylated can be, for example, a diseased cell, tissue or organ, an infected cell, tissue or organ, a damaged cell, tissue or organ, a cancerous cell, tissue or organ (e.g., a tumor cell within a tissue or organ), or a differentiating cell, tissue, or organ (e.g., a stem cell or a tissue or organ that comprises one or more stem cells). In some embodiments, the location wherein the RNA is differentially methylated is a cell, tissue, or organ that has been administered one or more drug(s), e.g., therapeutic drugs. Other locations that include differentially methylated RNA are known in the art.

In some embodiments, the location wherein the RNA is differentially methylated is a cancer cell. Non-limiting examples of cancers include: sarcomas, carcinomas, adrenocortical carcinoma, AIDS-related cancers, anal cancer, appendix cancer, astrocytomas, atypical teratoid/rhabdoid tumor, basal cell carcinoma, bladder cancer, brain stem glioma, brain tumors (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors, and pineoblastoma), breast cancer, bronchial tumors, cancer of unknown primary site, carcinoid tumor, carcinoma of unknown primary site, central nervous system atypical teratoid/rhabdoid tumor, central nervous system embryonal tumors, cervical cancer, childhood cancers, chordoma, colon cancer, colorectal cancer, craniopharyngioma, endocrine pancreas islet cell tumors, endometrial cancer, ependymoblastoma, ependymoma, esophageal cancer, esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioma, head and neck cancer, heart cancer, hypopharyngeal cancer, intraocular melanoma, islet cell tumors, Kaposi's sarcoma, kidney cancer, Langerhans cell histiocytosis, laryngeal cancer, lip cancer, liver cancer, lung cancer, malignant fibrous histiocytoma bone cancer, medulloblastoma, medulloepithelioma, melanoma, Merkel cell carcinoma, Merkel cell skin carcinoma, mesothelioma, metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, non-melanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pineal parenchymal tumors of intermediate differentiation, pineoblastoma, pituitary tumor, pleuropulmonary blastoma, primary hepatocellular liver cancer, prostate cancer, rectal cancer, renal cancer, renal cell (kidney) cancer, renal cell cancer, respiratory tract cancer, retinoblastoma, rhabdomyosarcoma, salivary gland cancer, Sezary syndrome, small cell lung cancer, small intestine cancer, soft tissue sarcoma, squamous cell carcinoma, squamous neck cancer, stomach (gastric) cancer, supratentorial primitive neuroectodermal tumors, testicular cancer, throat cancer, thymic carcinoma, thymoma, thyroid cancer, transitional cell cancer, transitional cell cancer of the renal pelvis and ureter, trophoblastic tumor, ureter cancer, urethral cancer, uterine cancer, uterine sarcoma, vaginal cancer, vulvar cancer, Waldenström macroglobulinemia, and Wilm's tumor.

In some embodiments, the cancer is an ovarian cancer, a lung cancer, a colon cancer, a leukemia, a prostate cancer, or a breast cancer.

In some embodiments, the location wherein the RNA is differentially methylated is a in a tissue, organ, or organ system affected by an autoimmune disease. The autoimmune disease can be, for example, rheumatoid arthritis, lupus, multiple sclerosis (MS), or psoriasis. In some embodiments, the tissue, organ, or organ system affected by an autoimmune disease is connective tissue, epithelial tissue, muscle tissue, or nervous tissue.

The analyte can be any suitable analyte described herein. In some embodiments, the analyte is a nucleic acid. In some embodiments, the analyte is RNA. In some embodiments, the analyte is messenger RNA (mRNA). In some embodiments, the RNA is expressed from the coding region of a gene. In some embodiments, the RNA is noncoding RNA (ncRNA), e.g., long ncRNA, such as long intergenic non-coding RNAs (lincRNAs).

In some embodiments, the RNA is a methylated RNA, e.g., an mRNA comprising one or more methylated adenosines. In some embodiments, the RNA is an unmethylated RNA, e.g., an RNA that does not comprise any methylated adenosine. In some embodiments, the RNA is an RNA that is differentially methylated in a location compared to a reference location.

In some embodiments, the methods provided herein comprise comparing the methylation status of an RNA analyte at a location in the tissue sample to the RNA methylation status of a reference location. In some embodiments, the reference location is a location in a healthy tissue, e.g., a healthy tissue corresponding to the tissue where the tissue sample is obtained. In some embodiments, the reference location is a location of non-cancerous tissue. In some embodiments, the reference location is a non-tumor tissue. In some embodiments, the reference location is a location with no abnormalities such as tumor, cancer, or disease. In some embodiments, the reference location is in a sample identified as having cancer. In some instances, the reference location is a location that includes a tumor.

In some embodiments, the methylation status of the RNA analyte in the tissue sample is different from the methylation status of an RNA analyte in a reference location. In some embodiments, the methylation of the RNA analyte in the tissue sample is higher than the methylation of an RNA analyte in a reference location. In some embodiments, the methylation of the RNA analyte in the tissue sample is lower than the methylation of an RNA analyte in a reference location. In some instances, the methylation status of a particular adenosine of an RNA analyte is different from an adenosine in an RNA analyte at a reference location. In some instances, a methyl group is located at a particular adenosine compared to a corresponding adenosine in the reference location. In some instances, a methyl group is not present at a particular adenosine compared to a methyl group present on an adenosine in the reference location. In some instances, the methylation status of the analyte includes at least 2, at least 3, at least 4, at least 5, at least 6, at least 7, at least 8, at least 9, at least 10, at least 20, at least 30, at least 40, at least 50, or more methyl groups compared to the corresponding adenosines in the reference location. In some instances, the methylation status of the RNA analyte does not include at least 2, at least 3, at least 4, at least 5, at least 6, at least 7, at least 8, at least 9, at least 10, at least 20, at least 30, at least 40, at least 50, or more methyl groups compared to the corresponding adenosines in the reference location.

The size of the analyte, e.g., RNA, can be any suitable size of a nucleic acid molecule in a biological sample. In some embodiments, the size of the target nucleic acid is about 50 nucleotides to about 100,000 nucleotides (e.g., about 50 nucleotides to about 200 nucleotides, about 200 nucleotides to about 500 nucleotides, about 500 nucleotides to about 1,000 nucleotides, about 1,000 nucleotides to about 2,000 nucleotides, about 2,000 nucleotides to about 4,000 nucleotides, about 4,000 nucleotides to about 6,000 nucleotides, about 6,000 nucleotides to about 8,000 nucleotides, about 8,000 nucleotides to about 10,000 nucleotides, about 10,000 nucleotides to about 20,000 nucleotides, about 20,000 nucleotides to about 30,000 nucleotides, about 30,000 nucleotides to about 40,000 nucleotides, about 40,000 nucleotides to about 50,000 nucleotides, about 50,000 nucleotides to about 60,000 nucleotides, about 60,000 nucleotides to about 70,000 nucleotides, about 70,000 nucleotides to about 80,000 nucleotides, about 80,000 nucleotides to about 90,000 nucleotides, or about 90,000 nucleotides to about 100,000 nucleotides.)

In some embodiments, identification of the methylated adenosines is indicative of the methylation status of the analyte, e.g., RNA. In some embodiments, the methylation status of the RNA analyte is indicated by the percentage of methylated adenosines over all adenosines in the RNA analyte. In some embodiments, the methods described herein further comprise comparing the determined sequence of the analyte with a reference sequence of the analyte, e.g., the sequence of the analyte without deamination. In some embodiments, the comparison comprises identifying guanosines in the determined sequence of the RNA analyte and comparing with the nucleotides in the corresponding positions in the reference sequence of the RNA analyte, and determining that one or more guanosines in the determined sequence using the methods described herein are unmethylated adenosines. In some embodiments, one or more adenosines in the analyte, e.g., RNA, can be methylated adenosines. In some embodiments, the determined sequence of the analyte comprises one or more adenosines, wherein the one or more adenosines are methylated adenosines. In some embodiments, about 0.0001% to about 50% (e.g., about 0.0001% to about 0.001%, about 0.001% to about 0.01%, about 0.01 to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.8% to about 0.9%, about 0.9% to about 1%, about 1% to about 2%, about 2% to about 3%, about 3% to about 4%, about 4% to about 5%, about 5% to about 6%, about 6% to about 7%, about 7% to about 8%, about 8% to about 9%, about 9% to about 10%, about 10% to about 15%, about 15% to about 20%, about 20% to about 25%, about 25% to about 30%, about 30% to about 35%, about 35% to about 40%, about 40% to about 45%, or about 45% to about 50%) of the adenosines in an RNA analyte are methylated. In some embodiments, no adenosine in an RNA analyte is methylated.

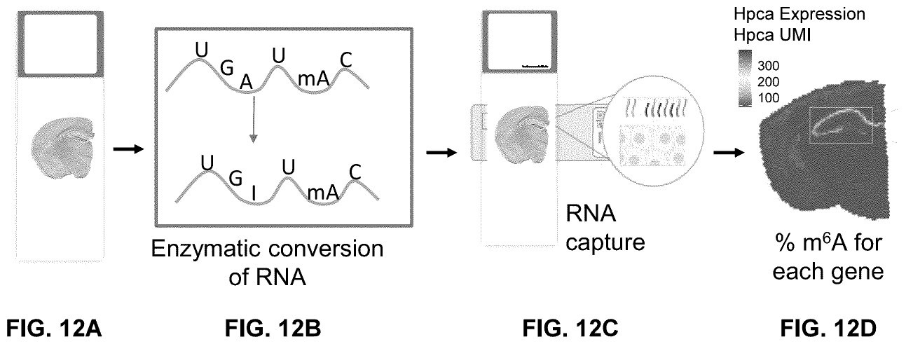

In some instances, the percentage or abundance of methylated adenosines can be determined and viewed as an image, such as a heat map as shown in FIG. 12D. In some instances, the map is generated based on the percentage of methylated adenosines or abundance of methylated adenosines by determining the location of the methylated adenosines using the spatial barcode in the capture probe.

The array can be any suitable array described herein. In some embodiments, the array is disposed on a slide. In some embodiments, the array includes a plurality of capture probes. In some embodiments, a 5′ end of a capture probe in the plurality is attached to the slide. In some embodiments, the array is a bead array. In some embodiments, a 5′ end of the capture probe is attached to a bead of the bead array. In some embodiments, the capture probe further comprises a unique molecular identifier (UMI). The UMI can be any suitable UMI described herein. In some embodiments, the UMI is positioned 5′ relative to the capture domain in the capture probe. In some embodiments, the capture probe further comprises a functional sequence. In some embodiments, the functional sequence is a primer sequence. In some embodiments, the primer sequence is used to extend the capture probe. In some embodiments, the adenosines in the UMI are methylated.

In some embodiments, the capture domain of the capture probe comprises a sequence that specifically binds to the capture probe binding domain of one or more probes. In some embodiments, the capture domain comprises at least one methylated adenosine. In some embodiments, the nucleotides in the capture domain are not deaminated.

The spatial barcode can be any suitable spatial barcodes described herein. In some embodiments, the spatial barcode comprises at least one methylated adenosine. In some embodiments, the nucleotides in the spatial barcode are not deaminated.

In some embodiments, the determining step comprises sequencing (i) all or a part of the sequence of the deaminated RNA analyte, or a complement thereof, and (ii) all or a part of the sequence of the spatial barcode, or a complement thereof. The sequencing can be performed using any suitable methods described herein. In some embodiments, the sequencing is high throughput sequencing. In some embodiments, the sequencing is sequencing by hybridization.

### C. Systems and Kits

Also disclosed herein are systems and kits used for any one of the methods disclosed herein. In some instances, the system of kit is used for analyzing an analyte in a biological sample.

In some instances, the system or kit includes a first substrate, wherein the first substrate comprises a plurality of capture probes, wherein a capture probe of the plurality of capture probes comprises: (i) a spatial barcode and (ii) a capture domain. In some instances, the system or kit includes a support device configured to retain a first substrate and a second substrate, wherein the biological sample is placed on the first substrate, and wherein the second substrate comprises a plurality of capture probes, wherein a capture probe of the plurality of capture probes comprises: (i) a spatial barcode and (ii) a capture domain.

In some instances, the system or kit includes a first probe and a second probe, wherein the first probe and the second probe each comprise a sequence that is substantially complementary to adjacent sequences of an analyte, wherein the second probe comprises a capture probe binding domain, and wherein the first probe and the second probe are capable of being ligated together to form a connected probe or ligated probe.

In some instances, the system or kit includes a plurality of analyte capture agents, wherein an analyte capture agent of the plurality of analyte capture agents comprises an analyte binding moiety and a capture agent barcode domain, wherein the analyte binding moiety specifically binds to the analyte, and wherein the capture agent barcode domain comprises an analyte binding moiety barcode and a capture handle sequence.

In some instances, the system of kit further includes an enzyme that modifies RNA. In some instances, the enzyme modifies non-methylated RNA. The enzyme can be an adenosine deaminase. In some instances, the enzyme is TadA or variant or functional equivalent thereof. In some instances, the TadA is TadA8.20.

In some instances, the system or kit further includes a first reagent medium for releasing capture probe from the substrate. In some instances, the system or kit includes a second reagent medium for permeabilizing the biological sample. Components of the first reagent medium and the second reagent medium are described throughout this application and are incorporated into this section. In some instances, the system or kit includes instructions for performing any one of the methods described herein.

In some instances, the second reagent medium includes a protease selected from pepsin or proteinase K. In some instances, the system or kit further includes an agent for releasing the ligated probe, such as an RNAse.

In some instances, the system or kit further includes an alignment mechanism on the support device to align the first substrate and the second substrate. In some instances, the alignment mechanism comprises a linear actuator and the first substrate comprises a first member and the second substrate comprises a second member. The linear actuator can be configured to move the second member along an axis orthogonal to the plane of the first member and/or the second member. The linear actuator can be configured to move the first member along an axis orthogonal to the plane of the first member and/or the second member. The linear actuator can be configured to move the first member, the second member, or both the first member and the second member at a velocity of at least 0.1 mm/sec. Finally, in some instances, the linear actuator can be configured to move the first member, the second member, or both the first member and the second member with an amount of force of at least 0.1 lbs.

### EXAMPLES

### Example 1

### Methods of Identifying Methylation Status of an RNA Analyte at a Location in a Biological Sample

FIGS. 12A-12D are exemplary workflows of the methods disclosed herein. As shown in FIG. 12A, the sample (e.g., a tissue section) is placed on a slide, stained, and imaged. The tissue is permeabilized, and a deaminating agent, e.g., a TadA enzyme, is applied to the tissue. During deamination, adenosines in the RNA within the tissue are globally deaminated and converted to inosine (I), while N6-methyladenosines in the RNA are not deaminated (see FIG. 12B).

In some instances, after the deaminating agent (e.g., deaminating enzyme) is washed away or neutralized, one or more probes targeting the RNA in the tissue are hybridized to the RNA to facilitate RNA capture (see FIG. 12C). After hybridization of the probes to the deaminated RNA, in one instance, one of the probes is extended using a polymerase, creating an oligonucleotide that is complementary to the regions between the hybridized probes. In another instance, two probes hybridize to adjacent sequences of the RNA. In both situations, the probes are ligated, creating a ligation product that includes a sequence complementary to the deaminated RNA. The ligation product (through one of the probes) includes a sequence (e.g., an overhang sequence or capture probe binding domain) complementary to a capture probe on a spatial array, which includes a plurality of capture probes, wherein each capture probe has a capture domain and a spatial barcode (see FIG. 12C). The ligation product is captured and the capture probe is extended using the ligation product as a template. In some embodiments, the ligation product is extended using the capture probe as a template, thereby generating an extended ligation product.

In some instances, after the deaminating agent is washed away or neutralized, the deaminated RNA is hybridized to capture probes on a spatial array. In some instances, this is achieved by hybridizing a portion of the deaminated RNA, e.g., a native or non-native poly(A) sequence, to a capture domain of the capture probe, e.g., a poly(T) sequence (see FIG. 12C). After capture of the deaminated RNA, the capture probe is extended using the deaminated RNA as a template, thereby generating an extended capture probe.

After capture of the ligation product or the deaminated RNA, the extended ligation products or complements thereof, the extended capture probes or complements thereof, are released from the spatial array or amplified prior to sequencing. After, the percentage or abundance of methylated adenosines in the RNA within the biological sample are determined. The data are viewed as an image, e.g., a heat map as shown in FIG. 12D. The heat map is generated based on the percentage of methylated adenosines or abundance of methylated adenosines detected in the sequencing data and attributing those methylated adenosines with a location in the spatial array using the spatial barcode in the capture probe.

### EMBODIMENTS

Embodiment (E)1. A method for identifying methylation status of RNA in a biological sample, the method comprising:
(a) contacting the biological sample with a first substrate;
(b) permeabilizing the biological sample;
(c) deaminating the RNA in the biological sample by converting one or more non-methylated adenosines in the RNA to inosines, thereby generating deaminated RNA;
(d) hybridizing the deaminated RNA to a capture domain of a capture probe affixed to an array, wherein the capture probe further comprises a spatial barcode; and
(e) determining sequences of (i) the spatial barcode, or a complement thereof, and (ii) all or part of the deaminated RNA, or a complement thereof, and using the determined sequences of (i) and (ii) to identify the methylation status of the RNA in the biological sample.

E2. A method for identifying methylation status of RNA in a biological sample, the method comprising:
(a) contacting the biological sample with a first substrate;
(b) permeabilizing the biological sample;
(c) deaminating the RNA in the biological sample by converting one or more non-methylated adenosines in the RNA to inosines, thereby generating deaminated RNA;
(d) contacting the biological sample with a plurality of probes comprising a first probe and a second probe, wherein the first probe or the second probe hybridize to the deaminated RNA and wherein:
the first probe and the second probe comprise a sequence complementary to a first sequence of the deaminated RNA; and
the second probe comprises a sequence complementary to a capture domain of a capture probe affixed to an array, wherein the capture probe further comprises a spatial barcode;
(e) ligating the first probe and the second probe, thereby generating a ligation product;
(f) hybridizing the ligation product to the capture domain of the capture probe; and
(g) determining sequences of (i) the spatial barcode, or a complement thereof, and (ii) all or part of the ligation product, or a complement thereof, and using the determined sequences of (i) and (ii) to identify the methylation status of the RNA in the biological sample.

E3. The method of E1 or E2, wherein the array is on the first substrate.

E4. The method of E1 or E2, wherein the array is on a second substrate.

E5. The method of E4, further comprising:
(i) aligning the first substrate with the second substrate, wherein a portion of the biological sample is aligned with at least a portion of the array; and
(ii) releasing the ligation product from the deaminated RNA.

E6. The method of any one of E2-E5, further comprising migrating the ligation product from the biological sample to the array.

E7. The method any one of E2-E6, wherein the first sequence of the deaminated RNA comprises inosine and/or adenosine, preferably wherein the adenosine is N6-methyladenosine.

E8. A method for determining the location of a methylated RNA molecule in a biological sample, the method comprising:
(a) contacting the biological sample with an adenosine deaminase such that one or more non-methylated adenosines in the RNA molecule is converted to inosine, thereby generating a deaminated RNA molecule;
(b) hybridizing the deaminated RNA molecule to a capture domain of a capture probe of an array comprising a plurality of capture probes, wherein the capture probe further comprises a spatial barcode;
(c) extending the capture probe using the deaminated RNA molecule as a template, thereby generating an extended capture probe; and
(d) determining sequences of (i) the spatial barcode, or a complement thereof, and (ii) all or part of the extended capture probe, or a complement thereof, and using the determined sequences of (i) and (ii) to determine the location of the methylated RNA molecule in the biological sample.

E9. A method for determining the location of a methylated RNA molecule in a biological sample, the method comprising:
(a) providing the biological sample mounted on a first substrate;
(b) contacting the biological sample with an adenosine deaminase such that one or more non-methylated adenosines in the RNA molecule is converted to inosine, thereby generating a deaminated RNA molecule;
(c) contacting the biological sample with a plurality of probes comprising a first probe and a second probe, wherein the first probe or the second probe hybridize to the deaminated RNA molecule and wherein:
the first probe and the second probe comprise a sequence complementary to a first sequence of the deaminated RNA molecule, wherein the first sequence of the deaminated RNA molecule comprises inosine and/or adenosine; and
the second probe comprises a sequence complementary to a capture domain of a capture probe of an array comprising a plurality of capture probes, wherein the capture probe further comprises a spatial barcode;
(d) ligating the first probe and the second probe, thereby generating a ligation product;
(e) hybridizing the ligation product to the capture domain of the capture probe; and
(f) determining sequences of (i) the spatial barcode, or a complement thereof, and (ii) all or part of the ligation product, or a complement thereof, and using the determined sequences of (i) and (ii) to determine the location of the methylated RNA molecule in the biological sample.

E10. The method of E9, wherein the array comprising a plurality of capture probes is on a second substrate, the method further comprising:
aligning the first substrate with the second substrate, wherein a portion of the biological sample is aligned with at least a portion of the array; and releasing the ligation product from the deaminated RNA molecule.

E11. The method of E10, further comprising migrating the ligation product from the biological sample to the array.

E12. The method of any one of E8-E11, wherein the methylated RNA molecule comprises one or more methylated adenosines, optionally wherein the one or more methylated adenosines is N6-methyladenosine.

E13. The method of E12, wherein the one or more methylated adenosines remains methylated upon contacting the biological sample with the adenosine deaminase.

E14. The method of any one of E8-E13 further comprising permeabilizing the biological sample.

E15. The method of E1 or E3, wherein the biological sample is disposed on the array.

E16. The method of any of one of E1-15, wherein the biological sample is optionally treated with a DNase.

E17. The method of any one of E1-E16, wherein the RNA or methylated RNA molecule is mRNA.

E18. The method of any one of E1-E7 and E15-E17, wherein the deaminating comprises treating the biological sample with an adenosine deaminase.

E19. The method of E18, wherein the adenosine deaminase is TadA or variant or equivalent thereof.

E20. The method of E19, wherein the adenosine deaminase is TadA8.20.

E21. The method of any one of E1-E20, wherein the capture probes comprise a poly(T) sequence.

E22. The method of any one of E1-E21, wherein capture probes further comprise one or more functional domains, a unique molecular identifier, a cleavage domain, or combinations thereof.

E23. The method of any one of E1-E22, wherein the biological sample is a tissue sample.

E24. The method of E23, wherein the tissue sample is a tissue section.

E25. The method of any one of E23-E24, wherein the tissue sample is a fixed tissue sample.

E26. The method of E25, wherein the fixed tissue sample is a formalin fixed paraffin embedded (FFPE) tissue sample.

E27. The method E26, wherein the FFPE tissue is deparaffinized and decrosslinked, optionally prior to step (a).

E28. The method of E25, wherein the fixed tissue sample is a formalin fixed paraffin embedded cell pellet.

E29. The method of any one of E23-E24, wherein the tissue sample is a fresh frozen tissue sample.

E30. The method of any one of E23-E29, wherein the tissue sample is fixed and stained, optionally prior to step (a).

E31. The method of E30, wherein the tissue sample is stained using immunofluorescence, immunohistochemistry, hematoxylin or eosin (H&E).

E32. The method of any one of E1-E31, wherein the determining comprises sequencing.

E33. The method of E34, wherein the sequencing comprises high-throughput sequencing.

E35. The method of any one of E1-E7 or E14-E34, wherein the permeabilizing comprises use of a permeabilization reagent comprising a protease.

E36. The method of E35, wherein the protease comprises pepsin.

E37. The method of E36, wherein the protease comprises proteinase K.

E38. The method of any one of E35-E37, wherein the permeabilization reagent further comprises one or more of a lipase, a DNase, an RNase, a detergent, or combinations thereof, preferably wherein the permeabilization reagent further comprises DNase.

E39. The method of any one of E2-E7 or E9-E29, further comprising extending the capture probe using the ligation product as a template, or extending the ligation product using the capture probe as a template, optionally wherein the extending comprises use of a polymerase.

E40. A kit comprising:
(a) an array comprising a plurality of capture probes, wherein each of the capture probes comprises a (i) a spatial barcode, (ii) a capture domain, and (iii) one or more functional domains; and
(b) an adenosine deaminase.

E41. The kit of E40, further comprising a polymerase.

E42. The kit of E41, wherein the polymerase comprises one of a T7 RNA polymerase, a T3 RNA polymerase, or an SP6 RNA polymerase.

E43. The kit of any one of E40-E42, wherein the kit further comprises one or more permeabilization reagents.

E44. The kit of E43, wherein the one or more permeabilization reagents comprise one or more of a protease, a lipase, a DNase, an RNase, a detergent, or combinations thereof.

E45. The kit of E44, wherein the protease comprises pepsin or proteinase K.

E46. A method for identifying methylation status of RNA in a biological sample, the method comprising:
(a) providing the biological sample mounted on a first substrate;
(b) contacting the biological sample with an adenosine deaminase such that one or more non-methylated adenosines in the RNA is converted to inosine, thereby generating a deaminated RNA;
(c) contacting the biological sample with a plurality of probes comprising a first probe and a second probe, wherein the first probe or the second probe hybridize to the deaminated RNA, and wherein the first probe and the second probe comprise a sequence complementary to a first sequence of the deaminated RNA molecule, wherein the first sequence of the deaminated RNA molecule comprises inosine and/or adenosine;
(d) ligating the first probe and the second probe, thereby generating a ligation product;
(e) hybridizing a padlock probe to the ligation product;
(f) circularizing the padlock probe using e.g., a ligase, thereby generating circularized padlock probe;
(g) optionally amplifying the circularized padlock probe, thereby generating an amplified padlock probe product;
(h) detecting the amplified padlock probe product using one or more detection probes, optionally wherein the one or more detection probes collectively comprise one or more fluorophores.

E47. A method for identifying methylation status of RNA in a biological sample, the method comprising:
(a) providing the biological sample mounted on a first substrate;
(b) contacting the biological sample with an adenosine deaminase such that one or more non-methylated adenosines in the RNA is converted to inosine, thereby generating a deaminated RNA;
(c) contacting the biological sample with a padlock probe, wherein the padlock probe hybridizes to the deaminated RNA;
(d) circularizing the padlock probe using, e.g., a ligase, thereby generating circularized padlock probe;
(e) optionally amplifying the circularized padlock probe, thereby generating an amplified padlock probe product; and
(f) detecting the amplified padlock probe product using one or more detection probes, optionally wherein the one or more detection probes collectively comprise one or more fluorophores.

E48. The method of E47, wherein the padlock probe comprises a first sequence at its 5′ end and a second sequence at its 3′ end, wherein the first sequence is substantially complementary to a first portion of the deaminated RNA, and the second sequence is substantially complementary to a second portion of the deaminated RNA.

E49. The method of E48, wherein the padlock probe further comprises a backbone sequence, optionally wherein the backbone sequence is disposed between the first sequence at its 5′ end and the second sequence at its 3′ end; optionally wherein a portion of the backbone sequence is substantially complementary to an amplification primer.

E50. The method of E49, wherein the backbone sequence comprises a backbone barcode sequence, optionally wherein the backbone barcode sequence is unique to and/or is used to identify the RNA.

E51. The method of any one of E48-E51, wherein circularizing the padlock probe comprises ligating the first sequence and the second sequence.

E52. The method of any one of E46-E51, wherein amplifying the circularized padlock probe comprises rolling circle amplification (RCA), optionally using a primer that is substantially complementary to a portion of the backbone sequence of the padlock probe.

E53. The method of any one of E46-E52, wherein the RNA comprises one or more methylated adenosines, optionally wherein the one or more methylated adenosines is N6-methyladenosine.
E54. The method of E53, wherein the one or more methylated adenosines remains methylated upon contacting the biological sample with the adenosine deaminase.

E55. The method of any one of E46-E54, wherein the RNA is mRNA.

E56. The method of any one of E46-E55, wherein the adenosine deaminase is TadA or variant or equivalent thereof, optionally wherein the adenosine deaminase is TadA8.20.

E57. The method of any one of E46-E56, wherein the biological sample is a tissue sample or tissue section, optionally a fixed tissue section or fresh frozen tissue section.

What is claimed is:

1. A method for identifying methylation status of an RNA molecule in a biological sample, the method comprising:
(a) deaminating the RNA molecule in the biological sample by converting one or more non-methylated adenosines in the RNA molecule to inosines, thereby generating deaminated RNA;
(b) hybridizing the deaminated RNA to a capture domain of a capture probe in a plurality of capture probes comprised in an array, wherein the capture probe further comprises a spatial barcode; and
(c) determining sequences of (i) the spatial barcode, or a complement thereof, and (ii) all or part of the deaminated RNA, or a complement thereof, and using the determined sequences of (i) and (ii) to identify the methylation status of the RNA molecule in the biological sample.

2. The method of claim 1, wherein the biological sample is disposed on the array, wherein the deaminating is performed with the biological sample disposed on the array.

3. The method of claim 1, wherein the RNA molecule is mRNA.

4. The method of claim 1, wherein the deaminating comprises treating the biological sample with an adenosine deaminase.

5. The method of claim 4, wherein the adenosine deaminase is a tRNA-specific adenosine deaminase (TadA) or variant or equivalent thereof.

6. The method of claim 4, wherein the adenosine deaminase is TadA8.20.

7. The method of claim 1, wherein capture probes further comprise one or more functional domains, a unique molecular identifier, a cleavage domain, or combinations thereof.

8. The method of claim 1, wherein the biological sample is a tissue section, optionally a fixed tissue section or fresh frozen tissue section.

9. The method of claim 1, wherein the determining comprises sequencing.

10. The method of claim 1, further comprising permeabilizing the biological sample, wherein the permeabilizing comprises use of a permeabilization reagent comprising a protease, a lipase, a DNase, an RNase, a detergent, or a combination thereof.

11. The method of claim 1, further comprising extending the capture probe using the deaminated RNA as a template, thereby generating an extended capture probe.

12. The method of claim 11, wherein the determining comprises determining a sequence of the extended capture probe, or an amplicon or a complement thereof.

13. The method of claim 1, wherein the RNA molecule comprises one or more methylated adenosines, wherein the one or more methylated adenosines is N⁶-methyladenosine, and optionally wherein the one or more methylated adenosines remains methylated upon contacting the biological sample with an adenosine deaminase.

14. The method of claim 4, wherein the biological sample is a formalin fixed paraffin embedded (FFPE) tissue section, the method further comprising decrosslinking the tissue section, incorporating a poly(A) sequence at a 3′ end of the deaminated RNA, or a combination thereof.

15. A method for identifying methylation status of RNA in a biological sample, the method comprising:
(a) providing the biological sample mounted on a first substrate;
(b) contacting the biological sample with an adenosine deaminase such that one or more non-methylated adenosines in the RNA is converted to inosine, thereby generating a deaminated RNA;
(c) contacting the biological sample with a padlock probe, wherein the padlock probe hybridizes to the deaminated RNA;
(d) circularizing the padlock probe using a ligase, thereby generating circularized padlock probe;
(e) optionally amplifying the circularized padlock probe, thereby generating an amplified padlock probe product; and
(f) detecting the amplified padlock probe product using one or more detection probes, optionally wherein the one or more detection probes collectively comprise one or more fluorophores.

16. The method of claim 15, wherein the padlock probe comprises a first sequence at its 5′ end and a second sequence at its 3′ end, wherein the first sequence is substantially complementary to a first portion of the deaminated RNA, and the second sequence is substantially complementary to a second portion of the deaminated RNA.

17. The method of claim 15, wherein the RNA comprises one or more methylated adenosines, optionally wherein the one or more methylated adenosines is N⁶-methyladenosine.

18. The method of claim 17, wherein the one or more methylated adenosines remains methylated upon contacting the biological sample with the adenosine deaminase.

19. The method of claim 15, wherein the adenosine deaminase is TadA or variant or equivalent thereof, optionally wherein the adenosine deaminase is TadA8.20.

20. The method of claim 15, wherein the biological sample is a tissue section, optionally a fixed tissue section or fresh frozen tissue section.
